(19) United States
(12) Patent Application Publication
(10) Pub. No.: US 2004/0033903 A1
(43) Pub. Date: Feb. 19, 2004

(54) Coated, agglomerated phytochemicals

(76) Inventors: Volker Kuellmer, Decatur, IL (US); Rishi Shukla, Decatur, IL (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/428,067

(22) Filed: May 2, 2003

Related U.S. Application Data
(60) Provisional application No. 60/376,836, filed on May 2, 2002.

(51) Int. Cl. A01N 25/28; A61K 9/16; A61K 9/50

(52) U.S. Cl. 504/359; 427/2.14; 424/490

(57) ABSTRACT

Coated and agglomerated products of materials containing phytochemicals, for example phytoestrogens such as isoflavones, and a method for the production of such coated and agglomerated products, are provided. The coating and agglomeration are accomplished using organic polymers in appropriate solvents, applied to the phytochemical-containing materials in a fluid bed system.

### CROSS-REFERENCE TO RELATED APPLICATIONS

This application claims the benefit of U.S. Provisional Application No. 60/376,836 filed May 2, 2002, the content of which is incorporated herein by reference.

### BACKGROUND OF THE INVENTION

1. Field of the Invention

The present invention relates to coated, agglomerated phytochemicals, and to a process for the coating and agglomeration of phytochemicals including isoflavones, lignans, and saponins.

2. Related Art

Currently, there is almost an epidemic of cancer, at least some of which is thought to be either caused or exacerbated by foods having a hormonal supplement derived from an animal origin. This thought is especially true for breast and prostate cancer. Other forms of cancers which are of special concern are skin cancer, colon cancer, urinary cancer, bladder and the like.

It is thought that many of those cancers, especially breast and prostate cancers, are either preventable or treatable by a use of phytochemicals, especially the phytoestrogens, and particularly isoflavones, as a source of supplemental hormones. As used herein, the term “isoflavone” includes malonyl, acetyl, glucoside, and aglycone forms of the isoflavones.

In addition to cancer, there are many other illnesses that may be treated by ingesting certain phytochemicals. Exemplary of these illnesses are: blood related illnesses such as excessive levels of cholesterol, coronary disease, abnormal blood lipid profiles and vascular effects; female symptoms; neurological symptoms such as migraine headaches, immunological symptoms, inflammations, dementia and alcoholism.

Plant materials are known to contain a number of classes of organic low molecular weight compounds that exert bioactivity in various animals. Historically, these compounds have been considered to be somewhat non-nutritive, however, recent scientific evidence now suggests these compounds may play an important role in the maintenance of health, in chemoprevention, and in the mitigation of certain conditions or diseases associated with the circulation of sex hormones, including sleep disorders and vaginal dryness.

Edible plants normally contained in the diet, or materials used as herbal remedies/dietary supplements, may contain collections of structurally related compounds. These related substances are often unique in their amounts and distribution when compared among various plant sources. The most notable groups of compounds exhibiting bioactivity are known as flavonoids, isoflavones, saponins, lignans, alkaloids, catechins and phenolic acids.

Epidemiology studies relating diet to disease suggest that dietary components may predispose populations to reduced risk of certain diseases. Far eastern populations consuming soy have reduced rates of breast, colon and prostate cancers and coronary heart disease, while populations in Finland have reduced rates of prostate cancer. Researchers are just now studying the specific compounds in the diet to understand the basis for the epidemiological observations.

Among the various plants consumed in the diet, several are rich sources of phytochemicals. Soy products contain high amounts of isoflavones and saponins. Unrefined diet grains include plants such as wheat, psyllium, rice, flax and oats that contain lignans. Cocoa contains catechins and phenolic acids. Certain non-dietary plants are also sources of the same chemical molecules, such as lignans and isoflavones in kudzu root or red clovers. Isoflavones and lignans act as weak estrogenic substances. Tea plants are also a rich source of phytochemicals, including catechins and phenolic acids.

Isoflavones can be used alone to treat or prevent breast cancer, prostate cancer, skin cancer, and colon cancer or as mechanism inhibitors. Isoflavones alone may also reduce or prevent various symptoms related to the onset and duration of menopause, including hot flashes and osteoporosis. Isoflavones alone may also be effective in certain cardiovascular applications, including heart disease, reducing cholesterol-lipid levels, modulating angiogenesis, and other vascular effects. Moreover, isoflavones alone have been implicated in reducing headaches, dementia, inflammation, and alcohol abuse, as well as immunomodulation.

Lignans have been implicated in preventing or treating breast cancer, prostate cancer, and colon cancer, as well as in reducing hot flashes, preventing osteoporosis, and as having antiviral potential. Lignans have also been shown to have antimitotic and fungicidal activity. A plant lignan, the catecholic nordihydro-guaiaretic acid, is a patent antioxidant once used in the food industry.

Saponins have been implicated in preventing or treating skin and colon cancer, in reducing serum cholesterol, and in immunomodulation and antiviral activity. Saponins also exhibit antioxidant effects and act as free radical scavengers.

People who eat a high soy diet show reduction of many of these above-discussed symptoms. This suggests that ingesting a combination of these phytochemicals in a ratio such as that found in soy may result in an additive or synergistic effect. However, a high soy diet has some undesirable effects, including flatulence, undesirable taste, and hesitancy among Western consumers to change their lifestyle to incorporate soy in their diets, even for such benefits.

Isoflavones, which are heterocyclic phenols, are understood to include the soy compounds genistin, daidzin and glycitein, as well as biochanin A, equol, formononetin, and odesmethylangolensin and natural derivatives thereof. These compounds and their aglycone or de-methylated aglycone forms, such as genistein and daidzein are believed to have similar activities once they are ingested. They are sometimes referred to as phytoestrogens.

Lignans are defined as compounds possessing a 2,3-dibenzylbutane structure. They include matairesinol, secoisolariciresinol, lariciresinol, isolariciresinol, nordihydroguaiaretic acid, pinoresinol, olivil, and other compounds that may be precursors of enterolactone and enterodiol, and modifications thereof, including diglucosides.

Phenolic acids include p-hydrobenzoic acid, protocatechuic acid, and vanillic acid. Other phenolic acids are chlorogenic acid, caffeic acid, ferulic acid, gallic acid, sinapic acid, syringic acid, coumaric acid, cinnamic acid, genistic acid, salicylic acid, hydroxy benzoic acid and hydroxy phenyl acetic acids and derivatives, including the various isomers and derivatives found in natural vegetable sources.

Catechins, or flavan 3-ols, include epigallocatechin, catechin, epicatechin and gallocatechin.

Saponins are the naturally occurring form of saponogenins, which are C-27 sterols in which the side chain has undergone metabolic changes to produce the spiroketal form. Saponins are the 3-O-glycosides of the parent steroid or triterpenes. U.S. Pat. No. 6,261,565 presents a discussion of various saponins.

Pinitol is a methyl ether of D-chiro-inositol and is readily hydrolyzed to D-chiro-inositol, and both pinitol and D-chiro-inositol have been shown to have potential benefits in the treatment of diabetes and various metabolic disorders, as well as a variety of additional diseases (see, e.g., U.S. Pat. No. 5,550,166).

Policosanols are long chain fatty alcohols of from about 24 to about 34 carbons. They are most typically extracted from sugar cane wax or bees wax, and may be effective in the treatment of high cholesterol, atherosclerosis, drug-induced gastric ulcers, and other syndromes (see, e.g., U.S. Pat. No. 5,856,316).

A proper diet contains the desired phytochemicals. However, a trouble is that many people do not have or do not like the proper kind of diet that provides the desirable effects. The problem is to furnish the necessary food values in some other form. Hence, there is a need for a process to refine phytochemicals such as phytoestrogens in a manner that addresses certain issues relating to the functionality of such products.

Products that contain phytochemicals, and especially isoflavone-containing products, that are currently marketed in bulk quantities often exhibit poor flow characteristics. These can be caused by such phenomena as interparticle ionic charge, or by the pick-up of moisture by hydroscopic components such as sugars and the like. Such phenomena can also cause, in addition to the poor flow characteristics, decreased long-term shelf life and other handling and product preparation issues. Furthermore, the high carbohydrate content of phytochemical-comprising products, such as isoflavone products, makes such products particularly difficult to tablet.

A number of patents (see, e.g., U.S. Pat. Nos. 5,925,381; 5,938,990; 6,001,554; 6,146,825; and 6,150,086) describe various aspects of a multiple micro-encapsulation system for oleophilic (fat-and-oil-soluble) substances, particularly the fat-soluble vitamins, comprising incorporating an oleophilic substance into a primary polymer, typically methylcellulose and hydroxypropyl methylcellulose. U.S. Pat. No. 6,162,474 describes a powder composition comprising droplets of a fat-soluble vitamin dispersed in a modified polysaccharide matrix, preferably modified starch. U.S. Pat. No. 6,139,872 describes a process for making a nutrient supplement powder by forming a plastic mass, extruding the material, coding and comminuting. U.S. Pat. No. 6,030,645 discloses a flowable dry particle consisting of at least one oleophilic substance as the active ingredient present in a matrix of at least one carrier material and a coating, the coating consisting of calcium silicate or a mixture of calcium silicate with one or more additional components.

International Patent Publication No. WO 97/38016 discloses cellulose esters which may be designed to dissolve under specific conditions and which may be used as coatings for controlled-release applications. U.S. patent application No. US2001/0009679 A1, discloses a powder composition containing at least one fat-soluble vitamin dispersed in a matrix of a natural polysaccharide gum or a mixture of gums having an emulsifying capacity and/or a protein or a mixture of proteins having an emulsifying capacity. International Patent Publication No. WO 01/80823 A2, discloses sol-gel microcapsules of inorganic polymers useful for the topical delivery of sensitive active ingredients.

For purposes of this disclosure, “phytochemicals” shall mean the phytoestrogens (e.g., isoflavones), lignans, catechins, phenolic acids, saponins, flavonoids, alkaloids, and other non-vitamin chemicals derived from plants. Due to the flow problems and shelf-life issues discussed above, a need exists in the art for a commercially useful coated, agglomerated phytochemicals, and a process for making the same, and particularly the isoflavones. Such coated phytochemical products would be useful for incorporation into nutraceutical supplement products, including tablets, supplement products for use in foods such as cereals or energy bars (for example), and other similar products.

### SUMMARY OF THE INVENTION

Therefore, the present invention provides a composition comprising free-flowing particulate phytochemicals, produced by a process of coating and agglomeration. The method of the invention involves the coating and agglomeration of a dried, phytochemical containing material with a polymer in a solvent, in a fluid bed system. The polymer, the solvent, and the process parameters used in the fluid bed system are all selected and/or manipulated by the skilled practitioner to produce a coated phytochemical end product having the desired particle size distribution and characteristics for the end use application of the coated phytochemical end product.

In one embodiment, soy isoflavones are coated and agglomerated using a water and/or alcohol soluble cellulose derivative (such as methylcellulose or ethylcellulose) in alcohol or an alcohol/water solvent. The coating and agglomeration are performed in a fluid bed system, using air pressures and evaporation temperatures in appropriate ranges to provide the desired particle size/agglomeration characteristics and the desired amount of coating on the particles.

The various objects and advantages of the present invention will be clear from the description that follows.

### DETAILED DESCRIPTION OF THE INVENTION

The present invention provides free flowing coated, agglomerated phytochemical comprising compositions, and most preferably isoflavone comprising compositions, and a process for their production. The coated and agglomerated phytochemical compositions of the invention can be used in various nutraceutical applications, including, for example, the production of dietary supplements, tablets (including multivitamin/mineral and/or nutraceutical tablets), foods, health bars, drinks, and the like.

The process of the invention involves the coating and agglomeration of phytochemical containing materials with a polymeric substance having appropriate characteristics for the particular application. The phytochemical comprising materials to be coated preferably comprise isoflavones, and more preferably, soy isoflavones. In other preferred embodiments, phytochemicals such as saponins, lignans, catechins, or phenolic acids may be coated and agglomerated. Phytochemicals can be extracted from numerous plant sources using various methods known in the art; for example, U.S. Pat. No. 5,702,752 teaches a process for extraction of isoflavones from soybeans. See also U.S. Pat. No. 6,261,565 for sources of other phytochemicals.

Various organic polymers are useful in the practice of the invention. The organic polymers suitable for use as coatings in the present invention include, but are not necessarily limited to, cellulose, water and/or alcohol soluble cellulose derivatives (methylcellulose and hydroxypropylmethylcellulose, for example), maltodextrin, alginic acid derivatives, calcium lactate, gum arabic, gelatin, sugar, sugar alcohols, glycerol, modified starches, pregelatinized starches, polyvinylpyrrolidones, stearic acid, gum acacia, cyclodextrin, lactose, maltodextrose, and even hydrogenated vegetable oil. See also, for additional examples, M. H. Gutcho, “Microcapsules and Microencapsulation Techniques.” Noyes Data Corp., Park Ridge, N.J., 1976. The preferred coating materials in the present invention are the water and/or alcohol soluble cellulose derivatives. Those of skill in the art will recognize that particular coatings may be more or less applicable to use in a particular application. Based on the present disclosure and the level of skill in the art, a skilled artisan will be able to modify the teachings herein and practice the invention in its various embodiments without undue experimentation.

By way of example, two particularly preferred coating agents, METHOCEL™ and ETHOCEL™ (Dow Chemical Co., Midland, Mich.), in their various forms, can produce coatings having various characteristics, including particle color, particle size, particle coarseness, etc., as well as having potential applicability in particular end uses. Furthermore, the various process parameters disclosed below will impact certain characteristics of the end-product coated, agglomerated particles. Again, those skilled in the art will be able to use the teachings herein to modify and adapt the present invention for the production of coated and agglomerated phytochemical comprising products having applicability to appropriate end uses without undue experimentation. Important aspects of the present invention include selection of the appropriate coating agent for the application in mind as discussed above, and selection of the appropriate solvent, as discussed further below.

Choice of solvent will, of course, vary depending upon the polymer chosen and the polymer's solubility in water and/or alcohol and/or organics, the size and/or viscosity of the polymer selected for coating, and the handling capabilities of the practitioner, among others. Particularly preferred solvents allow for control of drying/evaporation so that particles having desirable characteristics can be produced. For example, alcohols and/or alcohol/water solvents are preferred when using a water and/or alcohol soluble cellulose derivative such as methylcellulose or ethylcellulose. A particularly preferred alcohol for such an embodiment is ethanol, and in certain circumstances equally preferred will be ethanol/water mixtures, at various ratios (depending primarily upon the solubility of the polymer in water, and/or the size and/or viscosity of the polymer). For example, for ETHOCEL™ a 100% ethanol solvent is preferred, while for METHOCEL™ a mixture of 80% w/w ethanol: 20% w/w water may be preferred, again depending upon the size/viscosity chosen and the desired characteristics of the coated, agglomerated end product.

The fluid bed system and equipment used to carry out the coating and agglomeration process to produce the coated, agglomerated phytochemicals of the invention can ultimately be left to the practitioner's choice, but particular fluid bed-type systems are particularly preferred. One such system is the Vector FL-N form of Flo-Coater (Vector Corporation, 675 44^{th} Street, Marion, Iowa, 52302). Alternative systems are available; for example, the Wurster systems sold by Glatt Air Techniques Inc., Ramsey, N.J. The Vector system involves a top-spray system, whereas the Wurster systems sold by Glatt are modifications of conventional bottom-spray technology. Either type of system may be used in the process of making the compositions of the present invention. Another system that can be used to carry out the present invention is the Schugi Flex-O-Mix™ (Hosokawa Bepex, 333 N.E. Taft Street, Minneapolis, Minn. 55413).

As noted above, certain process parameters of the present invention can be manipulated in order to control final particle size/characteristics. For example, particle size (the amount of agglomeration) can in part be controlled by adjustment of the evaporation temperatures and/or by adjustment of nozzle air pressures (higher pressures producing smaller particles). As previously noted, the polymer and its physical characteristics (particularly its size) will also impact particle size.

The amount of coating applied can vary depending upon the ultimate goal of the coating process, and can be controlled through variation of amount of polymer used, process parameters, or by application of additional layers of the same or other polymers, as desired by the practitioner.

The coated particles of the invention are suitable for use in their free-flowing form, or they may also be tableted, using any number of art-recognized tableting processes and formulations. Such tablets are typically consumed as health/nutritional supplements. The free-flowing coated particles can be used in food or dairy applications, as well as in various health/nutraceutical formulations and application.

Having provided a general description of the invention, a more specific description is now provided by way of the following non-limiting examples.

### EXAMPLE 1

Isoflavone Coating

A 5% solution of METHOCEL™ or ETHOCEL™ (Dow Chemical Co., Midland, Mich.), in ethanol or a 80/20 (wt/wt) mixture of ethanol/water was used to agglomerate and coat isoflavone samples of batch 0112311 and 0201081 of NOVASOY® (Archer-Daniels-Midland Co., Decatur, Ill.) using a Vector FL-MI5-Fluid bed system (Vector Corp., Marion, Iowa). Lot. # 0112311:4 containers of 5 kg each; Lot. #0201081:2 container 5 kg each. Tables 1-8 contain process parameters, batch sizes, and coating chemicals used per trial. Calculations show that, theoretically, a 4% coating was applied. The color of agglomerated products is darker than the color of the starting materials, and the color is a function of coating agent used. ETHOCEL™ produces a lighter product than METHOCEL™. All process parameters listed in the enclosed protocols have a direct impact on product particle size distribution (see Table 9). An important aspect of the present invention is the right selection of the coating agent and solvent. METHOCEL™ has a higher binding power and consequently delivers a coarser product.

TABLE 1
(Trial No. 3)

Product: Novasoy ® Soy Isoflavones Lot No. 0112311
Spray Solution: Methylcellulose E-5 (5% Solution).
Nozzle diameter: 1.2 mm
Spray air pressure: 3 bar
Batch size: 4 kg with 5% solution of 200 gm Methylcellulose E-5 and 3800 gm
Ethanol.

| | | | Set | | | | STATIC | | PROCESS | |
|---|---|---|---|---|---|---|---|---|---|---|
| Time | PUMP | Grams | point | Inlet | Product | Exhaust | PRESSURE | | AIRFLOW | |
| min | g/min | applied | Temp | Temp | Temp | Temp | Inlet | Exhaust | Set pt. | Cfm |
| 0 | warm | 0 | 50 | 25.7 | 26.3 | 25.7 | 2.6 | 0.1 | 50 | 50 |
| 2 | | 0 | 50 | 29 | 27 | 26 | 2.6 | 0.1 | 50 | 47 |
| 0 | 50 | 0 | 50 | 30.8 | 22 | 25 | 2.6 | 0.1 | 50 | 50 |
| 5 | 40 | 222 | 50 | 42.1 | 23.7 | 24.6 | 5.5 | 0.4 | 80 | 77 |
| 11 | 40 | 474 | 50 | 50 | 33 | 28.8 | 7.7 | 0.4 | 80 | 84 |
| 17 | 40 | 716 | 50 | 50 | 34.5 | 30 | 8.9 | 0.6 | 100 | 103 |
| 26 | 40 | 1080 | 50 | 49 | 36.7 | 32.1 | 10.1 | 0.6 | 100 | 102 |
| 35 | 40 | 1452 | 50 | 50 | 37.3 | 33 | 9.5 | 0.6 | 100 | 104 |
| 40 | 40 | 1650 | 50 | 49.9 | 37.5 | 33.2 | 9.6 | 0.5 | 100 | 105 |
| 50 | 40 | 2058 | 50 | 49.9 | 37.9 | 33.8 | 9.1 | 0.5 | 100 | 101 |
| 63 | 40 | 2590 | 50 | 49.9 | 38.2 | 34.3 | 9.6 | 0.5 | 100 | 100 |
| 75 | 40 | 3078 | 50 | 50 | 38.2 | 34.5 | 11.1 | 0.5 | 100 | 103 |
| 78 | 40 | 3200 | 50 | 50 | 38.5 | 34.6 | 11.1 | 0.5 | 100 | 101 |
| 80 | 0 | 0 | 0 | 46 | 38.2 | 33.4 | 3.2 | 0.1 | 50 | 52 |

Final weight: 3.455 kg

TABLE 2
(Trial No. 4)

Product: Novasoy ® Soy Isoflavones Lot No. 0112311
Spray Solution: Ethylcellulose P-4 (4% Solution).
Nozzle diameter: 1.2 mm
Spray air pressure: 2.5 bar
Batch size: 4 kg with 5% solution of 200 gm Ethylcelluose P-4 and 3800 gm
Ethanol.

| | | | Set | | | | STATIC | | PROCESS | |
|---|---|---|---|---|---|---|---|---|---|---|
| Time | PUMP | Grams | point | Inlet | Product | Exhaust | PRESSURE | | AIRFLOW | |
| min | g/min | applied | Temp | Temp | Temp | Temp | Inlet | Exhaust | Set pt. | Cfm |
| 0 | warm | 0 | 50 | 24.4 | 24.4 | 25.3 | 24.8 | 0.1 | 50 | 50 |
| 2 | 0 | 0 | 50 | 24.3 | 24.3 | 25.2 | 24.4 | 0.1 | 50 | 50 |
| 0 | 50 | 0 | 50 | 28.9 | 25 | 21 | 5.1 | 0.1 | 50 | 50 |
| 7 | 50 | 352 | 50 | 38.1 | 25.7 | 21.2 | 6.1 | 0.4 | 82 | 80 |
| 15 | 50 | 764 | 50 | 49.7 | 30.1 | 23 | 4.1 | 0.5 | 82 | 80 |
| 17 | 55 | 862 | 50 | 50.1 | 31.6 | 24.1 | 4.1 | 0.4 | 81 | 80 |
| 22 | 55 | 1142 | 50 | 50.1 | 32.2 | 26.5 | 3.8 | 0.4 | 83 | 80 |
| 35 | 60 | 1858 | 50 | 50 | 33.3 | 29.7 | 7.1 | 0.5 | 82 | 80 |
| 43 | 70 | 2348 | 50 | 50.1 | 32.9 | 30 | 8.1 | 0.4 | 80 | 80 |
| 50 | 70 | 2820 | 50 | 49.9 | 31.6 | 29.4 | 8.4 | 0.5 | 83 | 80 |
| 56 | 70 | 3250 | 50 | 50 | 31.2 | 29.4 | 8.3 | 0.5 | 83 | 80 |
| 58 | 0 | 0 | 0 | 44.1 | 34 | 29.8 | 3.1 | 0.2 | 50 | 50 |
| 60 | 0 | 0 | 0 | 41.5 | 34.6 | 30 | 3.1 | 0.2 | 50 | 50 |

TABLE 3
(Trial No. 5)

Product: Novasoy ® Soy Isoflavones Lot No. 0112311
Spray Solution: Ethylcellulose P-10 (5% Solution).
Nozzle diameter: 1.2 mm
Spray air pressure: 3 bar
Batch size: 4 kg with 5% solution of 200 gm Ethylcellulose P-10 and 3800 gm
Ethanol.

| | | | Set | | | | STATIC | | PROCESS | |
|---|---|---|---|---|---|---|---|---|---|---|
| Time | PUMP | Grams | point | Inlet | Product | Exhaust | PRESSURE | | AIRFLOW | |
| min | g/min | applied | Temp | Temp | Temp | Temp | Inlet | Exhaust | Set pt. | Cfm |
| 0 | warm | 0 | 50 | 24.4 | 24.4 | 25.3 | 3.7 | 0.1 | 50 | 50 |
| 1 | | 0 | 50 | 40.2 | 29.6 | 26 | 3.7 | 0.1 | 50 | 50 |
| 0 | 80 | 0 | 50 | 38.9 | 24.8 | 24.7 | 4 | 0.2 | 88 | 80 |
| 6 | 80 | 464 | 55 | 52.1 | 25.8 | 23.4 | 4.4 | 0.4 | 83 | 80 |
| 10 | 80 | 774 | 55 | 55.8 | 28 | 24.5 | 3.8 | 0.4 | 81 | 80 |
| 15 | 80 | 1164 | 55 | 55.2 | 29.2 | 25.4 | 4.5 | 0.4 | 81 | 80 |
| 20 | 80 | 1574 | 52 | 55 | 30.2 | 26.4 | 6.7 | 0.5 | 80 | 80 |
| 30 | 80 | 2364 | 50 | 50.7 | 29.2 | 26.5 | 9.6 | 0.5 | 85 | 80 |
| 40 | 80 | 3270 | 50 | 49.9 | 28.6 | 26.1 | 9.2 | 0.4 | 83 | 80 |
| 45 | 0 | 0 | 50 | 49.8 | 36.8 | 28.5 | 3.2 | 0.3 | 49 | 50 |
| 55 | 0 | 0 | 50 | 50.5 | 40.4 | 31.1 | 3.2 | 0.3 | 52 | 50 |

Final weight: 4.070 kg
Loss on drying: 1.5%.

TABLE 4
(Trial No. 6)

Product: Novasoy ® Soy Isoflavones Lot No. 0112311
Spray Solution: Ethylcellulose P-10 (5% Solution).
Nozzle diameter: 1.2 mm
Spray air pressure: 3 bar
Batch size: 4 kg with 5% solution of 200 gm Ethylcellulose P-10 and 3800 gm
Ethanol.

| | | | Set | | | | STATIC | | PROCESS | |
|---|---|---|---|---|---|---|---|---|---|---|
| Time | PUMP | Grams | point | Inlet | Product | Exhaust | PRESSURE | | AIRFLOW | |
| min | g/min | applied | Temp | Temp | Temp | Temp | Inlet | Exhaust | Set pt. | Cfm |
| 0 | warm | 0 | 50 | 24.4 | 24.4 | 25.3 | 2.7 | 0.1 | 50 | 50 |
| 2 | | | 50 | 28 | 25.2 | 24.4 | 2.9 | 0.2 | 49 | 50 |
| 0 | 100 | 0 | 50 | 29.5 | 24 | 24.5 | 3.4 | 0.2 | 49 | 50 |
| 2 | 100 | 150 | 50 | 36.2 | 20 | 23.4 | 3.1 | 0.2 | 78 | 80 |
| 5 | 97 | 436 | 50 | 45.4 | 22.1 | 22.6 | 3.4 | 0.5 | 85 | 80 |
| 10 | 100 | 940 | 50 | 50.3 | 23.6 | 22.7 | 3.6 | 0.5 | 82 | 80 |
| 15 | 100 | 1440 | 50 | 50.1 | 23.9 | 22.8 | 3.7 | 0.5 | 83 | 80 |
| 20 | 100 | 1956 | 50 | 49.9 | 23.9 | 22.8 | 4.1 | 0.5 | 80 | 80 |
| 30 | 100 | 2940 | 50 | 49.9 | 24.1 | 22.9 | 4.4 | 0.5 | 83 | 80 |
| 33 | 100 | 3240 | 50 | 50.1 | 24.1 | 22.9 | 4.4 | 0.5 | 81 | 80 |
| 35 | 0 | 0 | 50 | 49.8 | 27.7 | 23.3 | 3.1 | 0.4 | 52 | 50 |
| 60 | 0 | 0 | 50 | 49.9 | 42.1 | 33.4 | 2.4 | 0.3 | 50 | 50 |

Final weight: 4.185 kg
Loss on drying: 1.6%.

TABLE 5
(Trial No. 7)

Product: Novasoy ® Soy Isoflavones Lot No. 0112311
Spray Solution: Methylcellulose E-15 (5% Solution).
Nozzle diameter: 1.2 mm
Spray air pressure: 2.5 bar
Batch size: 3.455 kg with 5% solution of 200 gm Methylcellulose E-15 and
3040 gm Ethanol and 760 gm of water.

| | | | Set | | | | STATIC | | PROCESS | |
|---|---|---|---|---|---|---|---|---|---|---|
| Time | PUMP | Grams | point | Inlet | Product | Exhaust | PRESSURE | | AIRFLOW | |
| min | g/min | applied | Temp | Temp | Temp | Temp | Inlet | Exhaust | Set pt. | Cfm |
| 0 | warm | 0 | 50 | 24.4 | 24.4 | 25.3 | 2.7 | 0.1 | 50 | 50 |
| 2 | | | 50 | 26.4 | 24.1 | 23.2 | 2.8 | 0.2 | 49 | 50 |
| 0 | 80 | 0 | 50 | 34.5 | 26.2 | 24.1 | 11 | 0.5 | 82 | 80 |
| 6 | 80 | 486 | 50 | 50.1 | 29.1 | 27.3 | 7.7 | 0.5 | 83 | 80 |
| 10 | 80 | 842 | 50 | 49.9 | 26.5 | 25.7 | 4.2 | 0.5 | 81 | 80 |
| 17 | 80 | 1386 | 50 | 49.9 | 25.8 | 24.6 | 3.7 | 0.5 | 80 | 80 |
| 22 | 80 | 2073 | 50 | 50 | 25.6 | 24.6 | 3.7 | 0.5 | 80 | 80 |
| 26 | 0 | 0 | 80 | 50.5 | 31.7 | 26.4 | 2.5 | 0.3 | 50 | 50 |
| 35 | 0 | 0 | 80 | 57.8 | 43.8 | 33.4 | 3.2 | 0.3 | 52 | 50 |
| 50 | 0 | 0 | 80 | 65.8 | 51.5 | 38.5 | 3.4 | 0.2 | 48 | 50 |

Loss on drying: 2.17%.

TABLE 6
(Trial No. 8)

Product: Novasoy ® Soy Isoflavones Lot No. 0112311
Spray Solution: Methylcellulose E-15 (5% Solution).
Nozzle diameter: 1.2 mm
Spray air pressure: 3 bar
Batch size: 4 kg with 5% solution of 200 gm Methylcellulose E-15 and 3040 gm
Ethanol and 760 gm of water.

| | | | Set | | | | STATIC | | PROCESS | |
|---|---|---|---|---|---|---|---|---|---|---|
| Time | PUMP | Grams | point | Inlet | Product | Exhaust | PRESSURE | | AIRFLOW | |
| min | g/min | applied | Temp | Temp | Temp | Temp | Inlet | Exhaust | Set pt. | Cfm |
| 0 | warm | 0 | 50 | 24.4 | 24.4 | 25.3 | 3.5 | 0.1 | 50 | 50 |
| 1 | 0 | 0 | 50 | 37.1 | 28.7 | 29 | 3.5 | 0.1 | 50 | 50 |
| 0 | 80 | 0 | 50 | 37.2 | 28.5 | 28.3 | 3.8 | 0.1 | 48 | 50 |
| 2 | 80 | 136 | 50 | 47.3 | 25.1 | 25.7 | 5.1 | 0.5 | 74 | 80 |
| 6 | 80 | 452 | 50 | 49.3 | 25.5 | 24.9 | 5.6 | 0.5 | 84 | 80 |
| 10 | 77 | 774 | 50 | 49.9 | 25.8 | 24.6 | 5.1 | 0.5 | 83 | 80 |
| 15 | 81 | 1179 | 50 | 50 | 25.8 | 24.3 | 4.3 | 0.4 | 80 | 80 |
| 26 | 80 | 2042 | 50 | 50 | 25.2 | 23.9 | 4.7 | 0.4 | 81 | 80 |
| 30 | 80 | 2372 | 50 | 50 | 24.9 | 23.6 | 4.7 | 0.4 | 82 | 80 |
| 41 | 80 | 3232 | 50 | 50 | 24.9 | 23.5 | 3.9 | 0.4 | 82 | 80 |
| 45 | 0 | 0 | 80 | 58.9 | 33.5 | 26.1 | 2.8 | 0.4 | 65 | 63 |
| 55 | 0 | 0 | 80 | 68 | 46.2 | 34.5 | 4.2 | 0.4 | 68 | 65 |
| 60 | 0 | 0 | 80 | 71.3 | 51.2 | 38.2 | 3.8 | 0.4 | 66 | 65 |
| 70 | 0 | 0 | 80 | 75.5 | 58.4 | 44.6 | 3.5 | 0.4 | 68 | 65 |

Final weight: 3.974 kg
Loss on drying: 2.32%

TABLE 7
(Trial No. 9)

Product: Novasoy ® Soy Isoflavones Lot No. 0112311
Spray Solution: Methylcellulose E-15 (5% Solution).
Nozzle diameter: 1.2 mm
Spray air pressure: 3 bar
Batch size: 4 kg with 5% solution of 200 gm Methylcellulose E-15 and 3040 gm
Ethanol and 760 gm of water.

| | | | Set | | | | STATIC | | PROCESS | |
|---|---|---|---|---|---|---|---|---|---|---|
| Time | PUMP | Grams | point | Inlet | Product | Exhaust | PRESSURE | | AIRFLOW | |
| min | g/min | applied | Temp | Temp | Temp | Temp | Inlet | Exhaust | Set pt. | Cfm |
| 0 | warm | 0 | 50 | 24.4 | 24.4 | 25.3 | 3.1 | 0.1 | 50 | 50 |
| 1 | | 0 | 50 | 23.5 | 24.2 | 22.7 | 3.1 | 0.1 | 48 | 50 |
| 0 | 80 | 0 | 50 | 23.4 | 24.5 | 22.9 | 3.4 | 0.2 | 50 | 50 |
| 2 | 80 | 88 | 50 | 25.2 | 21.4 | 23.1 | 3.8 | 0.5 | 70 | 80 |
| 6 | 80 | 314 | 50 | 47 | 25.6 | 24 | 8.1 | 0.5 | 80 | 80 |
| 11 | 80 | 710 | 50 | 50.5 | 26.1 | 24.6 | 6.4 | 0.6 | 84 | 80 |
| 16 | 80 | 1108 | 50 | 50.1 | 26.3 | 24.4 | 5.9 | 0.5 | 84 | 80 |
| 25 | 80 | 1826 | 50 | 49.9 | 25.8 | 24.4 | 3.6 | 0.5 | 83 | 80 |
| 30 | 80 | 2260 | 50 | 50 | 25.1 | 24.2 | 3.3 | 0.5 | 83 | 80 |
| 38 | 80 | 2870 | 50 | 50 | 25.3 | 24.1 | 3.6 | 0.5 | 82 | 80 |
| 42.5 | 80 | 3240 | 50 | 50.1 | 25.6 | 24.1 | 3.8 | 0.5 | 81 | 80 |
| 47 | 0 | 0 | 80 | 56.4 | 32.4 | 26 | 3.1 | 0.6 | 65 | 65 |
| 60 | 0 | 0 | 80 | 70.4 | 48.7 | 36.5 | 3.4 | 0.6 | 67 | 65 |
| 70 | 0 | 0 | 80 | 75.3 | 57 | 43.5 | 3.5 | 0.6 | 68 | 65 |

Final weight: 4.173 kg

TABLE 8
(Trial No. 10)

Product: Novasoy ® Soy Isoflavones Lot No. 0112311
Spray Solution: Methylcellulose E-15 (5% Solution).
Nozzle diameter: 1.2 mm
Spray air pressure: 3 bar
Batch size: 4 kg with 5% solution of 200 gm Methylcellulose E-15 and 3040 gm
Ethanol and 760 gm of water.

| | | | Set | | | | STATIC | | PROCESS | |
|---|---|---|---|---|---|---|---|---|---|---|
| Time | PUMP | Grams | point | Inlet | Product | Exhaust | PRESSURE | | AIRFLOW | |
| min | g/min | applied | Temp | Temp | Temp | Temp | Inlet | Exhaust | Set pt. | Cfm |
| 0 | warm | 0 | 50 | 24.4 | 24.4 | 25.3 | 2.8 | 0.1 | 50 | 50 |
| 1 | 0 | 0 | 50 | 36.6 | 31.8 | 36 | 3.1 | 0.2 | 49 | 50 |
| 0 | 100 | 0 | 50 | 39.4 | 32.8 | 35.4 | 3.5 | 0.3 | 48 | 50 |
| 2 | 100 | 184 | 50 | 47 | 29.4 | 33.8 | 3.5 | 0.4 | 70 | 80 |
| 5 | 100 | 410 | 50 | 51.5 | 29.6 | 30.1 | 5.4 | 0.5 | 75 | 80 |
| 10 | 98 | 876 | 50 | 50.1 | 29 | 29.2 | 5.2 | 0.6 | 82 | 80 |
| 15 | 90 | 1342 | 50 | 49.9 | 27.5 | 27.8 | 4.2 | 0.5 | 83 | 80 |
| 20 | 103 | 1843 | 50 | 49.9 | 25.5 | 26.4 | 3.5 | 0.6 | 83 | 80 |
| 25 | 100 | 2342 | 50 | 50 | 24.4 | 25.3 | 3.3 | 0.5 | 83 | 80 |
| 30 | 100 | 2886 | 50 | 49.9 | 24.4 | 24.8 | 3.4 | 0.5 | 80 | 80 |
| 34 | 100 | 3240 | 50 | 50 | 23.6 | 24.2 | 3.5 | 0.5 | 80 | 80 |
| 37 | 0 | 0 | 80 | 56 | 30.8 | 25.7 | 3.5 | 0.5 | 69 | 65 |
| 45 | 0 | 0 | 80 | 68.2 | 45 | 34.2 | 3.9 | 0.6 | 68 | 65 |
| 60 | 0 | 0 | 80 | 76.7 | 58.9 | 45.4 | 4 | 0.4 | 65 | 65 |

Final weight: 4.056 kg
Loss on drying: 2.63%

TABLE 9
Product particle size distribution

| | Trial # | | | | | | | |
|---|---|---|---|---|---|---|---|---|
| | 3 | 4 | 5 | 6 | 7(**) | 8 | 9 | 10 |
| Coating | Methocel | Ethocel | Ethocel | Ethocel | Methocel | Methocel | Methocel | Methocel |
| | E-5 | P-4 | P-10 | P-10 | E-15 | E-15 | E-15 | E-15 |
| Solvent: | Ethanol | Ethanol | Ethanol | Ethanol | 80/20* | 80/20* | 80/20* | 80/20* |
| Feed | 40 g/min. | 50-70 | 80 | 100 | 80 | 80 | 80 | 100 |
| rate: | | | | | | | | |
| % on | | | | | | | | |
| 20 | 0 | 0 | 0 | 0 | 0.4 | trace | trace | trace |
| mesh: | | | | | | | | |
| 40 | | | | | | | | 1.7 |
| mesh: | | | | | | | | |
| 80 | | | | | | | | 56.1 |
| mesh: | | | | | | | | |
| 100 | 13.5 | 12.7 | 12.6 | 13.5 | 62.1 | 53.0 | 32.6 | 25.8 |
| mesh: | | | | | | | | |
| 170 | | | | | | | | 10.8 |
| mesh: | | | | | | | | |
| 200 | 19.7 | 70.2 | 75.9 | 60.5 | 34.3 | 43.6 | 59.2 | 2.2 |
| mesh: | | | | | | | | |
| % in | 66.8 | 17.1 | 11.5 | 26.0 | 3.2 | 3.4 | 8.2 | 3.4 |
| pan: | | | | | | | | |

*80/20: 80% ethanol/20% water by weight

The products of Trial Numbers 8-10 are free flowing. No remaining electrostatic charge was detected through the usage of an 80/20 ethanol/water by weight mixture. Granulation and coating is one way to improve solid handling characteristics (batch process). Another option is to spray dry isoflavones to an estimated residual moisture of about 30% followed by agglomeration and drying in a fluid bed dryer (continuous process).

Product flowability was measured using an Aeroflow instrument from Amherst Process Instruments, Inc.

TABLE 10
Product flowability

Raw material 0201081 0112311
Lot #

| | | | Trial Number: | | | | | | | |
|---|---|---|---|---|---|---|---|---|---|---|
| | | | 3 | 4 | 5 | 6 | 7(**) | 8 | 9 | 10 |
| Time n | | | | | | | | | | |
| (seconds) | | | | | | | | | | |
| Avalanche | 0-45 | 0-27 | 0-71 | 0-95 | 0-100 | 0-127 | 0-103 | 0-107 | 0-119 | 0-126 |
| Range: | | | | | | | | | | |
| Number of | 46 | 28 | 72 | 96 | 101 | 128 | 104 | 108 | 120 | 127 |
| Avalanches: | | | | | | | | | | |
| Time between | | | | | | | | | | |
| Avalanches | | | | | | | | | | |
| Mean (sec): | 6.4 | 10.5 | 4.16 | 3.07 | 2.94 | 2.33 | 2.83 | 2.73 | 2.5 | 2.35 |
| Scatter(sec): | 3.78 | 9.05 | 1.18 | 0.99 | 1.98 | 1.07 | 1.7 | 1.52 | 1.24 | 1.17 |
| Maximum | 18 | 43 | 6.2 | 6 | 16 | 5.4 | 8.8 | 11 | 7.2 | 6.6 |
| (sec): | | | | | | | | | | |

(**)Product from trial 3 was used for the first METHOCEL ™ run to evaluate process parameter.

Analytical data showed that an isoflavone potency loss of only about 2% occurred through granulation and coating.

Tablet Trial

The following formulation was used:

Description: 50 mg Isoflavone tablets.
Batch Size: 5,000 tablets.

| Ingredient | | As | % w/w | mg/unit | g/batch |
|---|---|---|---|---|---|
| 50 mg | Isoflavones | Novasoy | 26.67 | 133.33 | 666.5 |
| | | (40% concentrate) | | | |
| | (filler) | Dicalcium | 59.62 | 298.12 | 1490.5 |
| | | phosphate | | | |
| | (binder) | Microcrystalline | 8.71 | 43.55 | 218.0 |
| | | cellulose | | | |
| | (disintegrant) | Sodium starch | 3.00 | 15.00 | 75.0 |
| | | glucolate | | | |
| | (flow agent) | Silicon dioxide | 1.00 | 5.00 | 25.0 |
| | (lubricant) | Magnesium | 1.00 | 5.00 | 25.0 |
| | | stearate | | | |

| | | | |
|---|---|---|---|
| Total: | 100.00 | | 2,500.0 |
| Total Tablet Weight: | | 500.00 mg | |

A 16 station Vector/Colton 2216 Single Rotary Press was used for the tableting trial (tooling: 15/32 inches round, 8 station plugged). The press was equipped with a pressure transducer to measure and record the compaction force.

Batch Size: 2.5 kg; Punches 8/20 rpm; 500 mg tablet; 160 tablets/min.,
run time ˜20 min.

| | | | | Isoflavone |
|---|---|---|---|---|
| Compression | Tablet | Tablet | Disintegration | content |
| Force (lbs.) | Hardness (kp) | Weight (mg) | Time (min) | (mg) |
| 3,500 | 10.0-11.3 | 499-502 | 5.0-5.5 | 60 |
| 5,000 | 13.2-14.2 | 500-502 | 7.5-8.5 | 60 |
| 7,000 | 17.8-19.0 | 500-502 | 14.5 | 60 |

Advantage: Tablets are not friable, good hardness, no tendency to cap (up to 7000 lbs. compression force), short disintegration time. Disadvantage: Surface color is not uniform. (Formula can be improved).

Having now fully described the present invention in some detail by way of illustration and example for purposes of clarity of understanding, it will be obvious to one of ordinary skill in the art that the same can be practiced by modifying or changing the invention with a wide and equivalent range of conditions, formulations and other parameters thereof, and that such modifications are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated herein by reference.

What is clamed is:

1. A process for producing a coated and agglomerated phytochemical composition comprising:
(a) dissolving an organic polymer in a solvent to produce a polymer-solvent solution;
(b) coating and agglomerating a phytochemical composition by contacting said phytochemical composition with said polymer-solvent solution to produce a coated and agglomerated phytochemical composition; and
(c) evaporating said solvent;
wherein said phytochemical composition is selected from the group consisting of isoflavones, phytoestrogens, flavonoids, saponins, lignans, alkaloids, catechins, phenolic acids, pinitols, policosanols, D-chiro-inositol, and mixtures thereof.

2. The process of claim 1, wherein said organic polymer comprises cellulose, ethylcellulose, methylcellulose, hydroxypropyl methylcellulose, water-soluble cellulose derivatives, alcohol-soluble cellulose derivatives, cyclodextrin, lactose, maltodextrose, maltodextrin, alginic acid derivatives, calcium lactate, gum arabic, gelatin, sugar, sugar alcohols, glycerol, modified starch, pregelatinized starch, polyvinylpyrrolidone, stearic acid, hydrogenated vegetable oil, gum acacia, or a mixture thereof.

3. The process of claim 2, wherein said organic polymer comprises a water-soluble cellulose derivative, an alcohol-soluble cellulose derivative, or a mixture thereof.

4. The process of claim 1, wherein said phytochemical composition (b) is coated and agglomerated in a fluid bed system.

5. The process of claim 1, wherein said solvent is evaporated at a temperature of about 20° C. to about 70° C.

6. The process of claim 1, wherein said phytochemical composition comprises one or more isoflavones.

7. The process of claim 6, wherein said phytochemical composition comprises one or more soy isoflavones.

8. The process of claim 4, wherein said polymer-solvent solution (b) contacts said phytochemical composition (b) by spraying said polymer-solvent solution.

9. The process of claim 8, wherein said polymer-solvent solution (b) is sprayed from the bottom of said fluid bed system.

10. The process of claim 8, wherein said polymer-solvent solution (b) is sprayed from the top of said fluid bed system.

11. The process of claim 1, wherein said phytochemical composition comprises one or more lignans.

12. The process of claim 11, wherein said phytochemical composition comprises one or more flax lignans.

13. The process of claim 3, wherein said organic polymer comprises ethylcellulose.

14. The process of claim 3, wherein said organic polymer comprises methylcellulose.

15. The process of claim 3, wherein said organic polymer comprises hydroxypropyl methylcellulose.

16. The process of claim 1, wherein said solvent (a) comprises alcohol.

17. The process of claim 16, wherein said solvent (a) further comprises water.

18. The process of claims 16 or 17, wherein said alcohol is ethanol.

19. The process of claims 16 or 17, wherein said alcohol is methanol.

20. The process of claim 17, wherein said solvent comprises about 80% alcohol and about 20% water.

21. The process of claim 1, wherein said polymer-solvent solution comprises ethylcellulose and ethanol.

22. The process of claim 1, wherein said polymer-solvent solution comprises methylcellulose and ethanol.

23. The process of claim 1, wherein said phytochemical composition (b) is dried prior to contacting said phytochemical composition with said polymer-solvent solution.

24. The process of claim 23, wherein said phytochemical composition (b) is spray dried.

25. The process of claim 1, wherein said coated and agglomerated phytochemical composition (b) is dried after said phytochemical composition contacts said polymer-solvent solution.

26. The process of claim 25, wherein said coated and agglomerated phytochemical composition (b) is dried in a fluid bed dryer.

27. The process of claim 6, wherein said coated and agglomerated phytochemical composition comprises at least about 25% isoflavones.

28. The process of claim 27, wherein said coated and agglomerated phytochemical composition comprises at least about 40% isoflavones.

29. The process of claim 1, wherein said phytochemical composition is contacted with said polymer-solvent solution at a rate from about 40 grams/minute to about 100 grams/minute.

30. The process of claim 29, wherein said phytochemical composition is contacted with said polymer-solvent solution at a pressure from about 0.5 bar to about 5 bar.

31. The process of claim 30, wherein said phytochemical composition is contacted with said polymer-solvent solution at a process air flow from about 50 cfm to about 120 cfm.

32. An edible product produced by the process of claim 1.

33. The product of claim 32, wherein said edible product is in a free-flowing form.

34. The product of claim 32, wherein said edible product is in a tablet form.

35. The product of claim 32, wherein said product is a beverage or pourable liquid.

36. The product of claim 32, wherein said product is a solid dry or semi-moist edible product.

37. A process for producing a coated and agglomerated phytochemical composition comprising:
(a) dissolving an organic polymer in a solvent to produce a polymer-solvent solution;
(b) adding a dry composition comprising one or more isoflavones to a fluid bed system;
(c) contacting said dry composition comprising one or more isoflavones with said polymer-solvent solution at a rate from about 40 grams/minute to about 100 grams/minute, at a pressure from about 0.5 bar to about 5 bar, and at a process air flow from about 50 cfm to about 120 cfm in order to produce a coated and agglomerated phytochemical composition; and
(d) evaporating said solvent at a temperature from about 20° C. to about 70° C.

38. The process of claim 37, wherein said polymer-solvent solution comprises ethylcellulose and ethanol.

39. The process of claim 37, wherein said polymer-solvent solution comprises methylcellulose and ethanol.

40. The process of claim 39, wherein said polymer-solvent solution further comprises about 20% water and about 80% ethanol.

41. The process of claim 37, wherein said polymer-solvent solution comprises hydroxypropyl methylcellulose.

42. The process of claim 37, wherein said coated and agglomerated phytochemical composition comprises at least 25% isoflavones.

43. The process of claim 42, wherein said coated and agglomerated phytochemical composition comprises at least 40% isoflavones.

44. An edible product produced by the process of claim 37.

45. The product of claim 44, wherein said edible product is in a free-flowing form.

46. The product of claim 44, wherein said edible product is in a tablet form.

47. The product of claim 44, wherein said product is a beverage or pourable liquid.

48. The product of claim 44, wherein said product is a solid dry or semi-moist edible product.
